(19) United States
(12) Patent Application Publication
(10) Pub. No.: US 2005/0196392 A1
(43) Pub. Date: Sep. 8, 2005

(54) Lesion repair polymerase compositions

(76) Inventor: Mark R. Andersen, San Mateo, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/061,099

(22) Filed: Feb. 18, 2005

Related U.S. Application Data
(60) Provisional application No. 60/546,549, filed on Feb. 20, 2004.

(51) Int. Cl. C12Q 1/68; A61K 38/47

(52) U.S. Cl. 424/94.61; 435/6

(57) ABSTRACT

Compositions comprising at least two polymerases, including a lesion repair polymerase, are provided. Methods for producing primer extension products using at least two polymerases, including a lesion repair polymerase, are also provided. Kits for producing primer extension products comprising at least two polymerases, including a lesion repair polymerase, are also provided.

This application claims the benefit of U.S. Provisional Application No. 60/546,549, filed Feb. 20, 2004, which is incorporated by reference herein for any purpose.

### FIELD

The disclosure generally relates to compositions comprising different polymerases and methods that employ such compositions.

### BACKGROUND

DNA polymerases are enzymes that synthesize DNA molecules from deoxynucleotide triphosphates (dNTPs) using a template DNA strand and a complementary oligonucleotide primer annealed to a portion of the template DNA strand. A detailed description of certain DNA polymerases and their characterization can be found, e.g., in Kornberg, DNA Replication Second Edition, W. H. Freeman (1989).

Y family DNA polymerases are capable of replicating damaged DNA and may be error-prone. Certain Y family DNA polymerases are described, e.g., in Goodman, Annu. Rev. Biochem. 71: 17-50 (2002); Boudsocq et al. DNA Repair 1:343-358 (2002); Woodgate Genes Dev. 13: 2191-2195 (1999); Vaisman et al. Mut. Res. 510: 9-22 (2002), and Yang, Curr. Opin. Struct. Biol. 13:23-30 (2003).

X family DNA polymerases are also capable of replicating damaged DNA and may be error prone. Certain X family DNA polymerases are described, e.g., in Zhang et al., J. Biol. Chem. 277(46): 44582-44587 (2002); Yang, Curr. Opin. Struct. Biol. 13:23-30 (2003); Aoufouchi et al. Nuc. Acids. Res. 28:3684-3693 (2000); Dominguez et al. EMBO J. 19:1731-1742 (2000); Garcia-Diaz et al. J. Mol. Biol. 301:851-867 (2000), and Havener et al. Biochem. 42:1777-1788 (2003).

DNA polymerases have a variety of uses in molecular biology techniques. Such techniques include primer extension reactions, DNA sequencing, genotyping, and nucleic acid amplification techniques such as the polymerase chain reaction (PCR).

### SUMMARY

In certain embodiments, a composition comprising at least one lesion repair polymerase and at least one second polymerase is provided. In certain embodiments, the composition further comprises a target nucleic acid. In certain embodiments, the target nucleic acid is a lesion-containing target nucleic acid. In certain embodiments, the composition further comprises at least one primer and at least one extendable nucleotide. In certain embodiments, the composition further comprises at least one of a terminator, a buffering agent, and an additive.

In certain embodiments, a method of amplifying a lesion-containing target nucleic acid is provided. In certain embodiments, the method comprises incubating the lesion-containing target nucleic acid with at least one primer, at least one extendable nucleotide, at least one lesion-repair polymerase, and at least one second polymerase under conditions to generate at least one primer extension product.

In certain embodiments, a method of sequencing a lesion-containing target nucleic acid is provided. In certain embodiments, the method of sequencing comprises incubating the lesion-containing target nucleic acid with at least one primer, at least one extendable nucleotide, at least one terminator, at least one lesion repair polymerase, and at least one second polymerase, under conditions to generate at least one primer extension product comprising a terminator.

In certain embodiments, the method of sequencing comprises forming a composition comprising the lesion-containing target nucleic acid, at least one primer, at least one extendable nucleotide, at least one lesion repair polymerase, and at least one second polymerase; and incubating the composition under conditions to generate a composition comprising at least one primer extension product; and incubating the composition comprising at least one primer extension product with at least one terminator to generate at least one primer extension product comprising a terminator.

In certain embodiments, the method of sequencing further comprises separating the at least one primer extension product comprising a terminator. In certain embodiments, the method further comprises detecting at least one of the at least one primer extension product comprising a terminator. In certain embodiments, the method further comprises determining the sequence of the lesion-containing target nucleic acid.

In certain embodiments, a method of genotyping a lesion-containing target nucleic acid is provided. In certain embodiments, the method comprises incubating the lesion-containing target nucleic acid with at least one primer, at least one extendable nucleotide, at least one lesion repair polymerase, and at least one second polymerase, under conditions to generate at least one primer extension product. In certain embodiments, the method further comprises separating the at least one primer extension product. In certain embodiments, the method further comprises detecting the at least one primer extension product. In certain embodiments, the method further comprises determining the genotype of the lesion-containing target nucleic acid.

In certain embodiments, a method of genotyping a lesion-containing target nucleic acid comprises incubating the lesion-containing target nucleic acid with at least one primer, at least one extendable nucleotide, at least one lesion repair polymerase, at least one second polymerase, and at least one probe under conditions to generate at least one primer extension product. In certain embodiments, a method of genotyping a lesion-containing target nucleic acid comprises forming a composition comprising the lesion-containing target nucleic acid, at least one primer, at least one extendable nucleotide, at least one lesion repair polymerase, and at least one second polymerase, and incubating the composition under conditions to generate at least one primer extension product; and incubating the at least one primer extension product with at least one probe.

In certain embodiments, the method of genotyping further comprises detecting at least one of the at least one probe. In certain embodiments, the method further comprises determining the genotype of the lesion-containing target nucleic acid.

In certain embodiments, a method of genotyping a lesion-containing target nucleic acid comprises incubating the lesion-containing target nucleic acid with at least one primer, at least one extendable nucleotide, at least one lesion repair polymerase, and at least one second polymerase under conditions to generate at least one primer extension product. In certain embodiments, the method further comprises separating the at least one primer extension product. In certain embodiments, the method further comprises incubating at least one of the at least one primer extension product with at least one probe. In certain embodiments, the method further comprises detecting at least one of the at least one probe. In certain embodiments, the method further comprises determining the genotype of the lesion-containing target nucleic acid.

In certain embodiments, a method of amplifying a lesion-containing target nucleic acid is provided. In certain embodiments, the method comprises incubating the lesion-containing target nucleic acid with at least one primer, at least one extendable nucleotide, at least one lesion repair polymerase, and at least one second polymerase, under conditions to generate at least one primer extension product. In certain embodiments, the method further comprises incubating the lesion-containing target nucleic acid with at least one intercalating dye.

In certain embodiments, the at least one second polymerase is not a lesion repair polymerase. In certain embodiments, at least one of the at least one second polymerase is thermostable. In certain embodiments, at least one of the at least one lesion repair polymerase is thermostable. In certain embodiments, at least one of the at least one lesion repair polymerase is an X family polymerase. In certain embodiments, the X family polymerase is selected from DNA polymerase β, DNA polymerase λ, DNA polymerase σ, DNA polymerase μ, DpoB, TDT, and ASFV polymerase X. In certain embodiments, at least one of the at least one lesion repair polymerase is a Y family polymerase. In certain embodiments, the Y family polymerase is selected from DNA polymerase η, DNA polymerase I, DNA polymerase κ, Rev 1, Rad 30, DinB, UmuC, UmuD2C, UmuD′2C, Dpo4, Dbh, and bacterial DNA pol II.

In certain embodiments, the at least one lesion repair polymerase is one lesion repair polymerase. In certain embodiments, the at least one second polymerase is one second polymerase. In certain embodiments, the lesion-repair polymerase and the second polymerase are present at a weight ratio from 1:4999 to 1:99. In certain embodiments, the weight ratio is 1:99 to 50:50. In certain embodiments, the weight ratio is 50:50 to 99:1. In certain embodiments, the lesion-repair polymerase and the second polymerase are present at a unit ratio from 1:4999 to 1:99. In certain embodiments, the unit ratio is 1:99 to 50:50. In certain embodiments, the unit ratio is 50:50 to 99:1.

In certain embodiments, the at least one second polymerase is two second polymerases. In certain embodiments, at least one of the two second polymerases is thermostable. In certain embodiments, the two second polymerases are Taq (G46D; F667Y; E681I) and Taq (G46D; F667Y; T664N; R660G). In certain embodiments, the unit ratio of Taq (G46D; F667Y; E681I) and Taq (G46D; F667Y; T664N; R660G) is from 1:99 to 50:50. In certain embodiments, the unit ratio is from 50:50 to 99:1. In certain embodiments, the weight ratio of the lesion-repair polymerase to the two second polymerases is from 1:4999 to 1:99. In certain embodiments, the weight ratio is from 1:99 to 50:50. In certain embodiments, the weight ratio is from 50:50 to 99:1. In certain embodiments, the unit ratio of the lesion-repair polymerase to the two second polymerases is from 1:4999 to 1:99. In certain embodiments, the unit ratio is from 1:99 to 50:50. In certain embodiments, the unit ratio is from 50:50 to 99:1.

In certain embodiments, a kit comprising at least one lesion repair polymerase and at least one second polymerase is provided. In certain embodiments, the kit comprises at least one X family polymerase. In certain embodiments, at least one of the at least one X family polymerase is selected from DNA polymerase β, DNA polymerase λ, DNA polymerase σ, DNA polymerase μ, DpoB, TDT, and ASFV polymerase X. In certain embodiments, the kit comprises at least one Y family polymerase. In certain embodiments, at least one of the at least one Y family polymerase is selected from DNA polymerase η, DNA polymerase I, DNA polymerase κ, Rev 1, Rad 30, DinB, UmuC, UmuD2C, UmuD′2C, Dpo4, Dbh, and bacterial DNA pol II. In certain embodiments, at least one of the second polymerases is thermostable. In certain embodiments, the kit comprises two second polymerases. In certain embodiments, the kit comprises Taq (G46D; F667Y; E681I) and Taq (G46D; F667Y; T664N; R660G). In certain embodiments, the kit further comprises at least one of a terminator, a buffering agent, a divalent cation, and an additive.

### DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the use of “or” means “and/or” unless specifically stated otherwise. Furthermore, the use of the term “including”, as well as other forms, such as “includes” and “included”, is not limiting.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

### CERTAIN DEFINITIONS

The term “nucleotide base” refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases, e.g., adenine, guanine, cytosine, uracil, and thymine, and analogs of the naturally occurring nucleotide bases, e.g., 7-deazaadenine, 7-deazaguanine, 7-deaza-8-azaguanine, 7-deaza-8-azaadenine, N6 -Δ2 -isopentenyladenine (6iA), N6 -Δ2 -isopentenyl-2-methylthioadenine (2ms6iA), N2 -dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propynylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38584), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide” refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2′-(C1-C6)alkoxyribose, 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1 -C6)alkylribose, 2′-deoxy-3′-(C1-C6)alkoxyribose and 2′-deoxy-3′-(C5 C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39352, and WO 99/14226). Exemplary LNA sugar analogs within a polynucleotide include, but are not limited to, the structures: 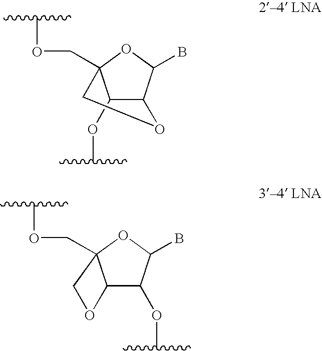
where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula: 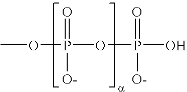
where α is an integer from 0 to 4. In certain embodiments, α is 2 and the phosphate ester is attached to the 3′- or 5′-carbon of the pentose. In certain embodiments, the nucleotides are those in which the nucleotide base is apurine, a 7-deazapurine, a pyrimidine, or an analog thereof. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group may include sulfur substitutions for the various oxygens, e.g. α-thio-nucleotide 5′-triphosphates. For a review of nucleotide chemistry, see, e.g., Shabarova, Z. and Bogdanov, A. Advanced Organic Chemistry of Nucleic Acids, VCH, New York, 1994.

The term “nucleotide analog” refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers which can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide. nucleic acids, in which the sugar phosphate backbone of the polynucleotide is replaced by a peptide backbone.

An “extendable nucleotide” is a nucleotide which is: (i) capable of being enzymatically or synthetically incorporated onto the terminus of a polynucleotide chain, and (ii) capable of supporting further enzymatic or synthetic extension. Extendable nucleotides include nucleotides that have already been enzymatically or synthetically incorporated into a polynucleotide chain, and have either supported further enzymatic or synthetic extension, or are capable of supporting further enzymatic or synthetic extension. Extendable nucleotides include, but are not limited to, nucleotide 5′-triphosphates, e.g., dNTP and NTP, phosphoramidites suitable for chemical synthesis of polynucleotides, and nucleotide units in a polynucleotide chain that have already been incorporated enzymatically or chemically.

The term “nucleotide terminator” or “terminator” refers to an enzymatically-incorporable nucleotide, which does not support incorporation of subsequent nucleotides in a primer extension reaction. A terminator is therefore not an extendable nucleotide. In certain embodiments, terminators are those in which the nucleotide is a purine, a 7-deaza-purine, a pyrimidine, or a nucleotide analog, and the sugar moiety is a pentose which includes a 3′-substituent that blocks further synthesis, such as a dideoxynucleotide triphosphate (ddNTP). In certain embodiments, substituents that block further synthesis include, but are not limited to, amino, deoxy, halogen, alkoxy and aryloxy groups. Exemplary terminators include, but are not limited to, those in which the sugar-phosphate ester moiety is 3′-(C1-C6)alkylribose-5′-triphosphate, 2′-deoxy-3′-(C1-C6)alkylribose-5′-triphosphate, 2′-deoxy-3′-(C1-C6)alkoxyribose-5-triphosphate, 2′-deoxy-3′-(C5-C14)aryloxyribose-5′-triphosphate, 2′-deoxy-3′-haloribose-5′-triphosphate, 2′-deoxy-3′-aminoribose-5′-triphosphate, 2′,3′-dideoxyribose-5′-triphosphate or 2′,3′-didehydroribose-5′-triphosphate. Terminators include, but are not limited to, T terminators, including ddTTP and dUTP, which incorporate opposite an adenine, or adenine analog, in a template; A terminators, including ddATP, which incorporate opposite a thymine, uracil, or an analog of thymine or uracil, in the template; C terminators, including ddCTP, which incorporate opposite a guanine, or guanine analog, in the template; and G terminators, including ddGTP and ddITP, which incorporate opposite a cytosine, or cytosine analog, in the template.

The term “label” refers to any moiety which can be associated with a molecule and: (i) provides a detectable signal; (ii) interacts with a second label to modify the detectable signal provided by the second label, e.g. FRET (Fluorescent Resonance Energy Transfer); (iii) stabilizes hybridization, e.g., duplex formation; or (iv) provides a member of a binding complex or affinity set, e.g., affinity, antibody/antigen, ionic complexation, hapten/ligand, e.g. biotin/avidin. Labeling can be accomplished using any one of a large number of known techniques employing known labels, linkages, linking groups, reagents, reaction conditions, and analysis and purification methods. Labels include, but are not limited to, light-emitting or light-absorbing compounds which generate or quench a detectable fluorescent, chemiluminescent, or bioluminescent signal (see, e.g., Kricka, L. in Nonisotopic DNA Probe Techniques (1992), Academic Press, San Diego, pp. 3-28). Fluorescent reporter dyes useful for labeling biomolecules include, but are not limited to, fluoresceins (see, e.g., U.S. Pat. Nos. 5,188,934; 6,008,379; and 6,020,481), rhodamines (see, e.g., U.S. Pat. Nos. 5,366,860; 5,847,162; 5,936,087; 6,051,719; and 6,191,278), benzophenoxazines (see, e.g., U.S. Pat. No. 6,140,500), energy-transfer fluorescent dyes (ETFDs), comprising pairs of donors and acceptors (see, e.g., U.S. Pat. Nos. 5,863,727; 5,800,996; and 5,945,526), and cyanines (see, e.g., Kubista, WO 97/45539), as well as any other fluorescent label capable of generating a detectable signal. Examples of fluorescein dyes include, but are not limited to, 6-carboxyfluorescein; 2′,4′,1,4,-tetrachlorofluorescein; and 2′,4′,5′,7′,1,4-hexachlorofluorescein. Labels also include, but are not limited to, semiconductor nanocrystals, or quantum dots (see, e.g., U.S. Pat. Nos. 5,990,479 and 6,207,392 B1; Han et al. Nature Biotech. 19: 631-635).

A class of labels are hybridization-stabilizing moieties which serve to enhance, stabilize, or influence hybridization of duplexes, e.g. intercalators and intercalating dyes (including, but not limited to, ethidium bromide and cyber green), minor-groove binders, and cross-linking functional groups (see, e.g., Blackburn, G. and Gait, M. Eds. “DNA and RNA structure” in Nucleic Acids in Chemistry and Biology, 2^{nd} Edition, (1996) Oxford University Press, pp.15-81). Yet another class of labels effect the separation or immobilization of a molecule by specific or non-specific capture, for example biotin, digoxigenin, and other haptens (see, e.g., Andrus, A. “Chemical methods for 5′ non-isotopic labeling of PCR probes and primers” (1995) in PCR 2: A Practical Approach, Oxford University Press, Oxford, pp. 39-54). Non-radioactive labelling methods, techniques, and reagents are reviewed in: Non-Radioactive Labelling, A Practical Introduction, Garman, A. J. (1997) Academic Press, San Diego.

Labels may be “detectably different”, which means that they are distinguishable from one another by at least one detection method. Detectably different labels include, but are not limited to, labels that emit light of different wavelengths, labels that absorb light of different wavelengths, labels that have different fluorescent decay lifetimes, labels that have different spectral signatures, labels that have different radioactive decay properties, labels of different charge, and labels of different size.

The term “labeled terminator” refers to a terminator that is physically joined to a label. The linkage to the label is at a site or sites on the terminator that do not prevent the incorporation of the terminator by a polymerase into a polynucleotide.

The term “target nucleic acid” refers to a nucleic acid sequence that serves as a template for a primer extension reaction. Target nucleic acids include, but are not limited to, genomic DNA, including mitochondrial DNA, chloroplast DNA and nucleolar DNA, cDNA, synthetic DNA, plasmid DNA, yeast artificial chromosomal DNA (YAC), bacterial artificial chromosomal DNA (BAC), and other extrachromosomal DNA, and primer extension products. Target nucleic acids also include, but are not limited to, RNA, synthetic RNA, mRNA, tRNA, and analogs of both RNA and DNA, such as peptide nucleic acids (PNA). In certain embodiments, target nucleic acids comprise one or more lesions.

Different target nucleic acids may be different portions of a single contiguous nucleic acid or may be on different nucleic acids. Different portions of a single contiguous nucleic acid may overlap.

A target nucleic acid may comprise one or more lesions. In certain embodiments, a target nucleic acid comprising one or more lesions is called a “lesion-containing target nucleic acid.” Lesions include, but are not limited to, one or more nucleotides with at least one abnormal alteration in its chemical properties, e.g., a base alteration, a base deletion, a sugar alteration, or an alteration which causes a strand break. Specifically, lesions include, but are not limited to, abasic sites; AAF adducts, including, but not limited to, N-(deoxyguanosine-8-yl)-2-acetylaminofluorene and N-(deoxyguanosine-8-yl)-2-aminofluorene; cis-cyn pyrimidine dimers (also referred to as cyclobutane pyrimidine dimers), including, but not limited to, cis-syn thymine-thymine dimers; 6-4 pyrimidine-pyrimidone dimers; benzo[a]pyrene diol epoxide adducts, including, but not limited to, benzo[a]pyrene diol epoxide deoxyadenosine adducts and benzo[a]pyrene diol epoxide deoxyguanosine adducts; oxidized guanine, including, but not limited to, 7,8-dihydro-8-oxoguanine, and 8-oxoguanine, (8-hydroxyguanine); oxidized adenine, including, but not limited to, 7,8-dihydro-8-oxoadenine, and 8-oxoadenine, (8-hydroxyadenine); 5-hydroxycytosine; 5-hydroxyuracil; 5,6-dihydouracil; cisplatin adducts, including but not limited to, 1,2-cisplatinated guanine; 5,6-dihydro-5,6-dihyroxythymine (thymine glycol); 1,N⁶-ethenodeoxyadenosine; O⁶-methylguanine; cyclodeoxyadenosine; 2,6-diamino-4-hydroxyformamidopyrimidine; 8-nitroguanine; N²-guanine monoadducts of 1,3-butadiene metabolites; and oxidized cytosine.

Lesions also include, but are not limited to, any alteration in a polynucleotide resulting from radiation, oxidative damage, and chemical mutagens. Sources of radiation include, but are not limited to, nonionizing radiation (e.g., UV radiation), or ionizing radiation (e.g., X-rays, gamma radiation, and corpuscular radiation (e.g., α-particle and β-particle radiation)). Sources of oxidative damage include, but are not limited to, oxidative damage mediated by one or more transition metals (e.g., the combination of H₂O₂ and CuCl₂)), and chemical mutagens. Chemical mutagens include, but are not limited to, base analogs (e.g., bromouracil or aminopurine), chemicals which alter the structure and pairing properties of bases (e.g., nitrous acid, nitrosoguanidine, methyl methanesulfonate (MMS), and ethyl methanesulfonate (EMS)), intercalating agents (e.g., ethidium bromide, acridine orange, and proflavin), agents altering DNA structure (e.g., large molecules that bind to bases in DNA and cause them to be noncoding (e.g., acetyl aminofluorene (AAF), N-acetoxy-2-aminofluorene (NAAAF), or cisplatin), agents causing inter- and intrastrand crosslinks (e.g., psoralens), methylated and acetylated bases, and chemicals causing DNA strand breaks (e.g., peroxides)).

The term “primer” refers to a polynucleotide or oligonucleotide that has a free 3′-OH (or functional equivalent thereof) that can be extended by at least one nucleotide in a primer extension reaction catalyzed by a polymerase. In certain embodiments, primers may be of virtually any length, provided they are sufficiently long to hybridize to a polynucleotide of interest in the environment in which primer extension is to take place. In certain embodiments, primers are at least 14 nucleotides in length. Primers may be specific for a particular sequence, or, alternatively, may be degenerate, e.g., specific for a set of sequences.

The terms “primer extension” and “primer extension reaction” are used interchangeably, and refer to a process of adding one or more nucleotides to a nucleic acid primer, or to a primer extension product, using a polymerase, a template, and one or more nucleotides.

A “primer extension product” is produced when one or more nucleotides has been added to a primer in a primer extension reaction. A primer extension product may serve as a target nucleic acid in subsequent extension reactions. A primer extension product may include a terminator. In certain embodiments, when a primer extension product includes a terminator, it is referred to as a “primer extension product comprising a terminator.”

As used herein, the terms “polynucleotide”, “oligonucleotide”, and “nucleic acid” are used interchangeably and refer to single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A polynucleotide may be composed entirely of deoxyribonucleotides, entirely of ribonucleotides, or chimeric mixtures thereof. The nucleotide monomer units may comprise any of the nucleotides described herein, including, but not limited to, nucleotides and nucleotide analogs. A polynucleotide may comprise one or more lesions. Polynucleotides typically range in size from a few monomeric units, e.g. 5-40 when they are sometimes referred to in the art as oligonucleotides, to several thousands of monomeric nucleotide units. Unless denoted otherwise, whenever a polynucleotide sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, and “T” denotes thymidine or an analog thereof, unless otherwise noted.

Polynucleotides may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below: 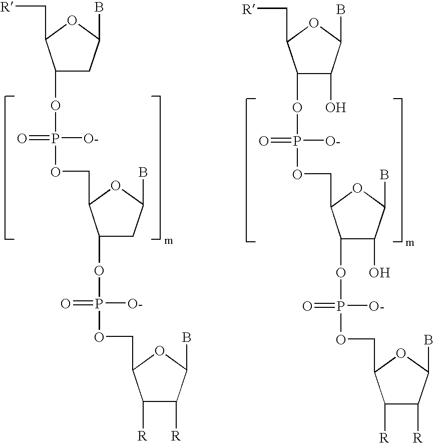
wherein each B is independently the base moiety of a nucleotide, e.g., a purine, a 7-deazapurine, a pyrimidine, or an analog thereof; each m defines the length of the respective nucleic acid and can range from zero to thousands, tens of thousands, or even more; each R is independently selected from the group comprising hydrogen, hydroxyl, halogen, —R″, —OR″, and −NR″R″, where each R″ is independently (C₁-C₆) alkyl or C₅ -C1₄) aryl, or two adjacent Rs may be taken together to form a bond such that the ribose sugar is 2′,3′-didehydroribose, and each R′ may be independently hydroxyl or 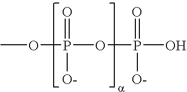
where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid”, “polynucleotide”, and “oligonucleotide” may also include nucleic acid analogs, polynucleotide analogs, and oligonucleotide analogs. The terms “nucleic acid analog”, “polynucleotide analog” and “oligonucleotide analog” are used interchangeably, and refer to a polynucleotide that contains at least one nucleotide analog and/or at least one phosphate ester analog and/or at least one pentose sugar analog. A polynucleotide analog may comprise one or more lesions. Also included within the definition of polynucleotide analogs are polynucleotides in which the phosphate ester and/or sugar phosphate ester linkages are replaced with other types of linkages, such as N-(2-aminoethyl)-glycine amides and other amides (see, e.g., Nielsen et al., 1991, Science 254:1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52: 4202); methylene(methylimino) (see, e.g., Vasseur et al., 1992, J. Am. Chem. Soc. 114: 4006); 3′-thioformacetals (see, e.g., Jones et al., 1993, J. Org. Chem. 58: 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate.

The term “microsatellite” refers to a repetitive stretch of a short sequence of DNA. In certain embodiments, the short sequence -of DNA is two bases in length. In certain embodiments, the short sequence of DNA is three bases in length. In certain embodiments, the short sequence of DNA is four bases in length. In certain embodiments, the short sequence of DNA is more than four bases in length. In certain embodiments, microsatellites include short tandem repeats (STRs). In certain embodiments, microsatellites can be used as genetic markers.

The term “genotype” refers to the specific allelic composition of one or more genes of an organism. The term “genotyping” refers to testing that reveals the specific alleles carried by an individual.

The terms “annealing” and “hybridization” are used interchangeably and refer to the base-pairing interaction of one nucleic acid with another nucleic acid that results in formation of a duplex, triplex, or other higher-ordered structure. In certain embodiments, the primary interaction is base specific, e.g., A/T and G/C, by Watson/Crick and Hoogsteen-type hydrogen bonding. Base-stacking and hydrophobic interactions may also contribute to duplex stability. The term “variant” refers to any alteration of a protein, including, but not limited to, changes in amino acid -sequence, substitutions of one or more amino acids, addition of one or more amino acids, deletion of one or more amino acids, and alterations to the amino acids themselves. In certain embodiments, the changes involve conservative amino acid substitutions. Conservative amino acid substitution may involve replacing one amino acid with another that has, e.g., similar hydrophobicity, hydrophilicity, charge, or aromaticity. In certain embodiments, conservative amino acid substitutions may be made on the basis of similar hydropathic indices. A hydropathic index takes into account the hydrophobicity and charge characteristics of an amino acid, and, in certain embodiments, may be used as a guide for selecting conservative amino acid substitutions. The hydropathic index is discussed, e.g., in Kyte et al., J. Mol. Biol., 157:105-131 (1982). It is understood in the art that conservative amino acid substitutions may be made on the basis of any of the aforementioned characteristics.

Alterations to the amino acids may include, but are not limited to, glycosylation, methylation, phosphorylation, biotinylation, and any covalent and noncovalent additions to a protein that do not result in a change in amino acid sequence. The term “amino acid” refers to any amino acid, natural or nonnatural, that may be incorporated, either enzymatically or synthetically, into a polypeptide or protein.

The term “polymerase” refers to an enzyme that is capable of adding at least one nucleotide onto the 3′ end of a primer that is annealed to a target nucleic acid. In certain embodiments, the nucleotide is added to the 3′ end of the primer in a template-directed manner. In certain embodiments, the polymerase is capable of sequentially adding two or more nucleotides onto the 3′ end of the primer. In certain embodiments, the polymerase is active at 37° C. In certain embodiments, the polymerase is active at a temperature other than 37° C. In certain embodiments, the polymerase is active at a temperature greater than 37° C. In certain embodiments, the polymerase is active at both 37° C. and other temperatures. A “DNA polymerase” catalyzes the polymerization of deoxynucleotides.

The term “lesion repair polymerase” refers to an enzyme that is capable of adding at least one nucleotide onto the 3′ end of a primer, or onto the 3′ end of a primer extension product, that is annealed opposite a lesion on a target nucleic acid comprising one or more lesions. In certain embodiments, the added nucleotide is a match for the template. In certain embodiments, the added nucleotide is a mismatch for the template. In certain embodiments, the target nucleic acid is not fully annealed to the primer, such that one or more nucleotides of the target nucleic acid are located within a bulge. In certain embodiments, the action of the lesion repair polymerase upon the target nucleic acid enables a second polymerase that cannot replicate a lesion-containing nucleic acid to replicate the target nucleic acid.

Lesion repair polymerases include, but are not limited to, X family polymerases and Y family polymerases. Certain exemplary X family polymerases include, but are not limited to, DNA polymerase β, DNA polymerase λ, DNA polymerase σ, DNA polymerase μ (also referred to as, e.g., pol μ), DpoB, TDT (also referred to as, e.g., terminal deoxynucleotidyltransferase), and DNA polymerase from African Swine Fever Virus (also referred to as, e.g., ASFV DNA polymerase X). Certain exemplary Y family polymerases include, but are not limited to, DNA polymerase η (also referred to as, e.g., XPV or RAD30A), DNA polymerase I (also referred to as, e.g., RAD30B), DNA polymerase κ (also referred to as, e.g., DinB1 or DNA polymerase IV), Rev1, Rad30 (also referred to as, e.g., DNA polymerase η), DinB (also referred to as, e.g., DNA polymerase IV), UmuC (also referred to as, e.g., DNA polymerase V or DNA polymerase V catalytic subunit), UmuD₂C (also referred to as, e.g., DNA polymerase V), UmuD′₂C (also referred to as, e.g., DNA polymerase V), Dpo4 (also referred to as, e.g., DNA polymerase IV), Dbh, and bacterial DNA pol II. X and Y family polymerases from many organisms are known in the art. Additional X and Y family polymerases can be identified by one skilled in the art.

The term “thermostable” refers to a polymerase that retains its ability to add at least one nucleotide onto the 3′ end of a primer that is annealed to a target nucleic acid at a temperature higher than 37° C. In certain embodiments, the thermostable polymerase remains active at a temperature greater than about 37° C. In certain embodiments, the thermostable polymerase remains active at a temperature greater than about 42° C. In certain embodiments, the thermostable polymerase remains active at a temperature greater than about 50° C. In certain embodiments, the thermostable polymerase remains active at a temperature greater than about 60° C. In certain embodiments, the thermostable polymerase remains active at a temperature greater than about 70° C. The term “non-thermostable polymerase” refers to a polymerase that does not retain its ability to add at least one nucleotide onto the 3′ end of a primer that is annealed to a target nucleic acid at a temperature higher than 37° C.

The term “unit” of polymerase is defined as the amount of polymerase that will catalyze the incorporation of 10 nmoles of total nucleotide into acid-insoluble form in 30 minutes. In certain embodiments, a unit is defined at the polymerase's optimum temperature. In certain embodiments, a unit of thermostable polymerase is defined at 74° C. In certain embodiments, a unit of non-thermostable polymerase is defined at 37° C. In certain embodiments, units are defined for specific reaction conditions.

In certain embodiments, the “unit ratio” of one polymerase to another polymerase in a composition is based on the percentage of the total units in the composition of each polymerase. In certain embodiments, a unit of each polymerase is defined under the same conditions. Thus, as a nonlimiting example, if the unit ratio of polymerase A to polymerase B is 60:40 and there are 10 total units of polymerase in the composition, then there are 6 units of polymerase A and 4 units of polymerase B.

In certain embodiments, the “weight ratio” of one polymerase to another polymerase in a composition is based on the percentage of the total weight of polymerases in the composition. Thus, as a nonlimiting example, if the weight ratio of polymerase A to polymerase B is 1:99 and there are 100 ng total polymerase in the composition, then there is 1 ng of polymerase A and 99 ng of polymerase B.

As used herein, “mobility-dependent analysis technique” or “MDAT” means an analytical technique based on differential rates of migration among different analyte types. In certain embodiments, the primer extension products may be separated based on, e.g., mobility, molecular weight, length, sequence, and/or charge. Any method that allows two or more nucleic acid sequences in a mixture to be distinguished, e.g., based on mobility, length, molecular weight, sequence and/or charge, is within the scope of the term MDAT. Exemplary mobility-dependent analysis techniques include, without limitation, electrophoresis, including gel or capillary electrophoresis; chromatography, including HPLC; mass spectroscopy, including MALDI-TOF;. sedimentation, including gradient centrifugation; gel filtration; field-flow fractionation; multi-stage extraction techniques; and the like. In certain embodiments, the MDAT is electrophoresis or chromatography.

As used herein, a “buffering agent” is a compound added to a composition of the invention which modifies the stability, activity, or longevity of one or more components of the composition by regulating the pH of the composition. Non-limiting exemplary buffering agents include Tris and Tricine.

As used herein, an “additive” is a compound added to a composition which modifies the stability, activity, or longevity of one or more components of the composition. In certain embodiments, an additive inactivates contaminant enzymes, stabilizes protein folding, and/or decreases aggregation. Exemplary additives include, but are not limited to, glycerol, DMSO, dithiothreitol (DTT), Thermoplasma acidophilum inorganic pyrophosphatase (TAP), and bovine serum albumin (BSA).

### CERTAIN EXEMPLARY EMBODIMENTS OF THE INVENTION

In certain embodiments, the present invention is directed to compositions and methods for generating at least one primer extension product. According to certain embodiments, the present invention provides methods for generating a primer extension product using at least two polymerases. In certain embodiments, the methods use at least one lesion-repair polymerase and at least one second polymerase. In certain embodiments, the methods employ compositions comprising at least one target nucleic acid, at least one primer, at least one extendable nucleotide, at least one lesion-repair polymerase, and at least one second polymerase. In certain embodiments, at least one of the at least one target nucleic acid comprises one or more lesions. In certain embodiments, a duplex (double stranded polynucleotide) is formed between a target nucleic acid and a primer in the composition. In certain embodiments, the primer hybridizes to a predetermined location on the target nucleic acid.

In certain embodiments, the composition is incubated under appropriate reaction conditions, such that one or more extendable nucleotides are incorporated sequentially onto the 3′ end of the primer. In certain embodiments, the incubation step is thermocycling. In certain embodiments, the thermocycling constitutes a PCR reaction. PCR reactions and methods of carrying out PCR are described, e.g., in Current Protocols in Molecular Biology, Ausubel et al., eds., Wiley Interscience Publishers (2003), ch. 15, “The Polymerase Chain Reaction.”

In certain embodiments, the composition is first incubated at an optimum temperature for at least one polymerase in the composition and then incubated at an optimum temperature for at least one other polymerase in the composition. In certain embodiments, one or more of the polymerases are added between the first incubation and the second incubation. In certain embodiments, the composition comprises at least one lesion-repair polymerase during the first incubation. In certain embodiments, the composition is first incubated at 37° C. In certain embodiments, the composition is then subjected to thermocycling. In certain embodiments, the thermocycling constitutes a PCR reaction. In certain embodiments, the primer extension products generated by the primer extension reaction may then be separated based on size.

Polymerases may or may not be thermostable. In certain embodiments, the composition comprises at least one thermostable polymerase. In certain embodiments, the composition comprises at least one non-thermostable polymerase. In certain embodiments, the composition comprises at least one lesion-repair polymerase. In certain embodiments, the composition, comprises at least one thermostable polymerase and at least one. lesion-repair polymerase. In certain embodiments, the composition comprises at least one non-thermostable polymerase and at least one lesion-repair polymerase. In certain embodiments, the composition comprises at least one thermostable polymerase, at least one non-thermostable polymerase, and at least one lesion-repair polymerase. In any of these embodiments, at least one lesion-repair polymerase can be thermostable. In any of these embodiments, at least one lesion-repair polymerase can be non-thermostable.

Exemplary thermostable polymerases include, but are not limited to, Thermus thermophilus HB8 (described, e.g., in U.S. Pat. No. 5,789,224); mutant Thermus thermophilus HB8, including, but not limited to, Thermus thermophilus HB8 (D18A; F669Y; E683R), Thermus thermophilus HB8 (Δ271; F669Y; E683W), and Thermus thermophilus HB8 (D1 8A; F669Y); Thermus oshimai (described, e.g., in U.S. Provisional Application No. 60/334,798, filed Nov. 30, 2001, corresponding to U.S. application No. 20030194726, Thermus oshimai Nucleic Acid Polymerases, published Oct. 16, 2003); mutant Thermus oshimai, including, but not limited to, Thermus oshimai (G43D; F665Y); Thermus scotoductus (described, e.g., in U.S. Provisional Application No. 60/334,489, filed Nov. 30, 2001); mutant Thermus scotoductus, including, but not limited to, Thermus scotoductus (G46D; F668Y); Thermus thermophilus 1B21 (described, e.g., in U.S. Provisional Application No. 60/336,046, filed Nov. 30, 2001), mutant Thermus thermophilus 1B21, including, but not limited to; Thermus thermophilus 1B21 (G46D; F669Y); Thermus thermophilus GK24 (described, e.g., in U.S. Provisional Application No. 60/336,046, filed Nov. 30, 2001); mutant Thermus thermophilus GK24, including, but not limited to, Thermus thermophilus GK24 (G46D; F669Y); Thermus aquaticus polymerase; mutant Thermus aquaticus polymerase, including, but not limited to, Thermus aquaticus (G46D; F667Y) (AmpliTaq®) FS or Taq (G46D; F667Y), described, e.g., in U.S. Pat. No. 5,614,365), Taq (G46D; F667Y; E681I), and Taq (G46D; F667Y; T664N; R660G); Pyrococcus furiosus polymerase; mutant Pyrococcus furiosus polymerase; Thermococcus gorgonarius polymerase; mutant Thermococcus gorgonarius polymerase; Pyrococcus species GB-D polymerase; mutant Pyrococcus species GB-D polymerase; Thermococcus sp. (strain.9°N-7) polymerase; mutant Thermococcus sp. (strain 9°N-7) polymerase; Bacillus stearothermophilus polymerase; mutant Bacillus stearothermophilus polymerase; Tsp polymerase; mutant Tsp polymerase; ThermalAce™ polymerase (Invitrogen); Thermus flavus polymerase; mutant Thermus flavus polymerase; Thermus litoralis polymerase; mutant Thermus litoralis polymerase. In certain embodiments, a thermostable polymerase is a mutant of a naturally-occurring polymerase.

Exemplary non-thermostable polymerases include, but are not limited to DNA polymerase I; mutant DNA polymerase I, including, but not limited to, Klenow fragment and Klenow fragment (3′→5′ exonuclease minus); T4 DNA polymerase; mutant T4 DNA polymerase; T7 DNA polymerase; mutant T7 DNA polymerase; phi29 DNA polymerase; and mutant phi29 DNA polymerase.

Lesion repair polymerases include, but are not limited to, members of the Y family of polymerases and members of the X family of polymerases.

Exemplary members of the X family of polymerases include, but are not limited to, DNA polymerase λ from, e.g., mouse, human, cow, sheep, and Arabidopsis thaliana; DNA polymerase a from, e.g., human; DNA polymerase p from, e.g., human and mouse; DpoB, from, e.g., human, mouse, zebrafish, soybean, and Paramecium tetraurelia; TDT from, e.g., human, dog, cow, opossum, mouse, chicken, salamander, trout, zebrafish, nurse shark, and Neurospora crassa; and DNA polymerase from African Swine Fever Virus (also referred to as, e.g., ASFV DNA polymerase X).

In certain embodiments, additional X family polymerases may be identified by sequence homology and/or structural homology to one or more known X family polymerases. In certain embodiments, X family polymerases comprise a minimal nucleotidyltransferase (MNT) core domain. In certain embodiments, an MNT core domain comprises a poorly-conserved N-terminal α-helix, followed by a four-strand β-sheet with a short α-helix inserted between strands 1 and 2, and a variable helix placed at different angles in different members of the family after strand 4. See, e.g., Aravind et al., Nucl. Acids Res., 27:1609-1618 (1999) and Holm et al., Trends in Biochem. Sci., 20: 345-347 (1995).

Exemplary Y family polymerases include, but are not limited to, DNA polymerase η from, e.g., human, mouse, chicken, yeast, C. elegans, Arabidopsis thaliana, Anopheles gambiae, Oryza sativa, and D. melanogaster;DNA polymerase i from, e.g., human, mouse, rat, yeast, D. melanogaster, Neurospora crassa, Silurana tropicalis, Anopheles gambiae, Ictalurus punctatus, and Danio rerio; DNA polymerase κ from, e.g., human, mouse, rat, chicken, yeast, C. elegans; Rev1 from, e.g., human, mouse, D. melanogaster, Neurospora crassa, and yeast; Rad30 from, e.g., yeast and Arabidopsis thaliana; DinB from, e.g., Bordetella pertussis, Bacillus subtilis, Rhizobium meliloti, Halobacterium species NRC-1 and E. coli; DNA polymerase IV from, e.g., Thermoanaerobacter tengcongensis, Vibrio vulnificus, Vibrio parahaemolyticus, Rhizobium meliloti, Vibrio cholerae, Pseudomonas aeruginosa, Pasteurella multocida, Yersinia pestis, Ralstonia solanacearum, Streptococcus pyogenes, Streptococcus pneumoniae, Clostridium acetobutylicum, Ureaplasma parvum, Neisseria meningitides, Lactococcus lactis, Staphylococcus aureus, Corynebacterium glutamicum, E. coli, Salmonella typhimurium, Bacillus subtilis, Bacillus cereus, Bacillus anthracis, Streptomyces coelicolor, Listeria innocua, Listeria monocytogenes, Clostridium perfringens, crenarchaeote 4B7, Escherichia fergusonli, Brucella melitensis, Xanthomonas axonopodis, Xanthomonas campestris, Caulobacter vibrioides, Fusobacterium nucleatum, Mycobacterium tuberculosis, Mycobacterium bovis, Methanosarcina mazei, Agrobacterium tumefaciens, Methanosarcina acetivorans, Mesorhizobium loti, and Sulfolobus tokodaii; UmuC, UmuD₂C, and UmuD′₂C from, e.g., E. coli, Chromobacterium violaceum, Prochlorococcus marinus, Leishmania major, Citrobacter freundii, Synechococcus, Bacillus subtilis, Shewanella oneidensis, Salmonella enterica, Salmonella typhimurium, Mycoplasma gallisepticum, Nitrosomonas europaea, Shigella flexneri, Lactobacillus plantarum, Synechocystis, Proteus vulgaris, Xanthomonas campestris, Lactococcus lactis, Shigella flexneri, and Streptococcus pneumoniae; Dpo4 from,e.g., Sulfolobus solfataricus P2; Dbh from, e.g., Sulfolobus solfataricus P1; and DNA pol II from, e.g., E. coli.

Additional exemplary Y family polymerases may be identified by sequence homology and/or structural homology to one or more known Y family polymerases. In certain embodiments, Y family polymerases have a polydactyl right-handed architecture. See, e.g., Trincao et al., Mol. Cell, 8: 417-426 (2001). In certain embodiments, the polydactyl right-handed structure comprises a palm domain, a fingers domain, a thumb domain, and a polymerase-associated domain (PAD). In certain embodiments, the palm domain comprises a large subdomain and a small subdomain. In certain embodiments, the large subdomain comprises a mixed 6-stranded β sheet flanked by two long a helices. In certain embodiments, the large subdomain is similar to the large subdomain in certain other DNA polymerases, such as T7 polymerase and Taq polymerase. In certain embodiments, the small subdomain comprises a cluster of α helices. In certain embodiments, the fingers domain and/or the thumb domain of a Y family polymerase is/are smaller relative to the fingers domain and/or the thumb domain of certain other DNA polymerases, such as T7 polymerase. In certain embodiments, the PAD domain (residues 393-508 of S. cerevisiae Polη) comprises a mixed β sheet and two α helices. The PAD domain is not found in certain non-lesion repair DNA polymerases, such as T7 polymerase and Taq polymerase.

In certain embodiments, Y family DNA polymerases contain five conserved sequence motifs, designated I to V. See, e.g., FIG. 3 of Trincao et al., Mol. Cell, 8: 417-426 (2001). In certain embodiments, motifs I and III map to the palm domain, motif II is part of the fingers domain on the left side of the palm, motif IV forms a helix lying atop the palm domain on the right side, and motif V is part of the thumb domain. See, e.g., Trincao et al., Mol. Cell, 8: 417-426 (2001); Johnson et al., Mol. Cell. Biol., 23: 3008-3012 (2003); and references cited therein. In certain embodiments, catalytic residues are found in motifs I and III (e.g., Asp30, Asp155, and Glu156 in yeast Polη).

In certain embodiments, polymerases have mutations that reduce discrimination against 3′-dideoxynucleotide terminators as compared with nucleotide triphosphates. In certain embodiments, a polymerase bearing one or more of these mutations may incorporate 3′-deoxynucleotide terminators with greater efficiency than does the wild type polymerase (see, e.g., U.S. Pat. No. 5,885,813 and U.S. Pat. No. 6,265,193). In certain embodiments, mutations that reduce discrimination against 3′-dideoxynucleotide terminators are in the nucleotide-binding region of the polymerase. In certain embodiments, the nucleotide-binding region is located from about amino acid 520 to about amino acid 832 of the polymerase.

In certain embodiments, polymerases have mutations that reduce discrimination against fluorescent-labeled nucleotides. In certain embodiments, a polymerase bearing one or more of these mutations may incorporate fluorescent-labeled nucleotides with greater efficiency than does the wild type polymerase (see, e.g., U.S. Pat. No. 5,885,813 and U.S. Pat. No. 6,265,193). In certain embodiments, mutations that reduce discrimination against fluorescent-labeled nucleotides are in the nucleotide-binding region of the polymerase.

In certain embodiments, polymerases have mutations that reduce discrimination against ETFD-labelled terminators.

In certain embodiments, DNA polymerases possess exonuclease activity that may allow them to remove incorporated 3′-deoxynucleotide terminators. In certain embodiments, a mutant polymerase bearing one or more mutations or deletions may have reduced 3′-5′ exonuclease activity. In certain embodiments, such mutations or deletions are made in the amino-terminal region of the DNA polymerase. Certain examples of such mutations and deletions are described, e.g., in U.S. Pat. No. 4,795,699; U.S. Pat. No. 5,541,099; and U.S. Pat. No. 5,489,523. In certain embodiments, such mutations or deletions are made in the region of DNA polymerase that confers 3′-5′ exonuclease activity. In certain embodiments, that region is located from about amino acid 1 to about amino acid 272 of the DNA polymerase.

In certain embodiments, a composition comprises at least one lesion-repair polymerase. In certain embodiments, the at least one lesion-repair polymerase is selected from DNA polymerase rl, DNA polymerase I, DNA polymerase K, Rev1, Rad30, DinB, UmuC, UmuD₂C, UmuD′₂C, Dpo4, Dbh, and bacterial DNA pol II. In certain embodiments, the composition comprises at least one second polymerase. In certain embodiments, the at least one second polymerase is a non-lesion repair polymerase. In certain embodiments, at least one of the at least one second polymerase is thermostable. In certain embodiments, at least one of the at least one second polymerase is non-thermostable.

In certain embodiments, the composition comprises two polymerases. In various embodiments, the two polymerases may be present in a composition at any unit ratio. In various embodiments, the two polymerases may be present in a unit ration of between 1:4999 and 50:50. In certain embodiments, the two polymerases are present in a composition at a unit ratio of 1:4999. In certain embodiments, the unit ratio is 1:1999. In certain embodiments, the unit ratio is 1:999. In certain embodiments, the unit ratio is 1:500. In certain embodiments, the unit ratio is 1:99. In certain embodiments, the unit ratio is 5:95. In certain embodiments, the unit ratio is 10:90. In certain embodiments, the unit ratio is 20:80. In certain embodiments, the unit ratio is 30:70. In certain embodiments, the unit ratio is 40:60. In certain embodiments, the unit ratio is 50:50.

In various embodiments, the two polymerases may be present in a composition at any weight ratio. In various embodiments, the two polymerases are present in a weight ratio of between 1:4999 and 50:50. In certain embodiments, the two polymerases are present in a composition at a weight ratio of 1:4999. In certain embodiments, the weight ratio is 1:1999. In certain embodiments, the weight ratio is 1:999. In certain embodiments, the weight ratio is 1:99. In certain embodiments, the weight ratio is 5:95. In certain embodiments, the weight ratio is 10:90. In certain embodiments, the weight ratio is 20:80. In certain embodiments, the weight ratio is 30:70. In certain embodiments, the weight ratio is 40:60. In certain embodiments, the weight ratio is 50:50.

In certain embodiments, the composition comprises three polymerases. In certain embodiments, the composition comprises three or more polymerases, wherein each of the three or more polymerases is independently selected from an X family polymerase, a Y family polymerase, and a polymerase that is neither an X family nor a Y family polymerase. In various embodiments, the three polymerases may be present in any unit ratio or in any weight ratio. In certain embodiments, the composition comprises more than three polymerases.

In certain embodiments, the composition comprises Taq (G46D; F667Y; E681I), Taq (G46D; F667Y; T664N; R660G), and at least one lesion-repair polymerase.

In certain embodiments, the combination of Taq (G46D; F667Y; E681I) and Taq (G46D; F667Y; T664N; R660G) is referred to as FS-I/FS-NG. In various embodiments, Taq (G46D; F667Y; E681I) and Taq (G46D; F667Y; T664N; R660G) in FS-I/FS-NG may be at any unit ratio. In various embodiments, Taq (G46D; F667Y; E681I) and Taq (G46D; F667Y; T664N; R660G) in FS-I/FS-NG may be at any weight ratio.

In various embodiments, the unit ratio of Taq (G46D; F667Y; E681I) and Taq (G46D; F667Y; T664N; R660G) in FS-I/FS-NG is between 99:1 and 1:99. In certain embodiments, the unit ratio of Taq (G46D; F667Y; E681I) and Taq (G46D; F667Y; T664N; R660G) in FS-I/FS-NG is 2:1. In certain embodiments, the unit ratio of Taq (G46D; F667Y; E681I) and Taq (G46D; F667Y; T664N; R660G) in FS-I/FS-NG is 99:1. In certain embodiments, the unit ratio of Taq (G46D; F667Y; E681I) and Taq (G46D; F667Y; T664N; R660G) in FS-I/FS-NG is 90:10. In certain embodiments, the unit ratio of Taq (G46D; F667Y; E681I) and Taq (G46D; F667Y; T664N; R660G) in FS-I/FS-NG is 80:20. In certain embodiments, the unit ratio of Taq (G46D; F667Y; E681I) and Taq (G46D; F667Y; T664N; R660G) in FS-I/FS-NG is 70:30. In certain embodiments, the unit ratio of Taq (G46D; F667Y; E681I) and Taq (G46D; F667Y; T664N; R660G) in FS-I/FS-NG is 60:40. In certain embodiments, the unit ratio of Taq (G46D; F667Y; E681I) and Taq (G46D; F667Y; T664N; R660G) in FS-I/FS-NG is 50:50. In certain embodiments, the unit ratio of Taq (G46D; F667Y; E681I) and Taq (G46D; F667Y; T664N; R660G) in FS-I/FS-NG is 40:60. In certain embodiments, the unit ratio of Taq (G46D; F667Y; E681I) and Taq (G46D; F667Y; T664N; R660G) in FS-I/FS-NG is 30:70. In certain embodiments, the unit ratio of Taq (G46D; F667Y; E681I) and Taq (G46D; F667Y; T664N; R660G) in FS-I/FS-NG is 20:80. In certain embodiments, the unit ratio of Taq (G46D; F667Y; E681I) and Taq (G46D; F667Y; T664N; R660G) in FS-I/FS-NG is 10:90. In certain embodiments, the unit ratio of Taq (G46D; F667Y; E681I) and Taq (G46D; F667Y; T664N; R660G) in FS-I/FS-NG is 1:99.

In certain embodiments, FS-I/FS-NG can be combined with at least one lesion-repair polymerase. In certain embodiments, at least one of the at least one lesion repair polymerase is selected from DNA polymerase λ, DNA polymerase σ, DNA polymerase μ, DpoB; TDT, and ASFV DNA polymerase X. In certain embodiments, at least one of the at least one lesion-repair polymerase is selected from DNA polymerase η, DNA polymerase I, DNA polymerase κ, Rev1, Rad30, DinB, UmuC, UmuD₂C, UmuD′₂C, Dpo4, Dbh, and bacterial DNA pol II. In certain embodiments, the at least one lesion-repair polymerase is at least one X-family polymerase and at least one Y-family polymerase.

In various embodiments, FS-I/FS-NG and the at least one lesion-repair polymerase may be combined at any unit ratio. In various embodiments, FS-I/FS-NG and the at least one lesion-repair polymerase may be combined at any weight ratio. In various embodiments, FS-I/FS-NG and the at least one lesion-repair polymerase are combined at a weight ratio of between 1:99 and 4999:1. In certain embodiments, FS-I/FS-NG and the at least one lesion-repair polymerase are combined at a weight ratio of 1:99. In certain embodiments, the weight ratio is 10:90. In certain embodiments, the weight ratio is 20:80. In certain embodiments, the weight ratio is 30:70. In certain embodiments, the weight ratio is 40:60. In certain embodiments, the weight ratio is 50:50. In certain embodiments, the weight ratio is 60:40. In certain embodiments, the weight ratio is 70:30. In certain embodiments, the weight ratio is 80:20. In certain embodiments, the weight ratio is 90:10. In certain embodiments, the weight ratio is 99:1. In certain embodiments, the weight ratio is 999:1. In certain embodiments, the weight ratio is 1999:1. In certain embodiments, the weight ratio is 4999:1.

In certain embodiments, a target nucleic acid can be amplified using the polymerase chain reaction (PCR). PCR is described, e.g., in Current Protocols in Molecular Biology, Ausubel et al., eds., Wiley Interscience Publishers (2003), ch. 15, “The Polymerase Chain Reaction.”

In certain embodiments, a lesion repair polymerase repairs a target nucleic acid comprising one or more lesions before amplification, sequencing, and/or genotyping of the target nucleic acid. In certain embodiments, a lesion repair polymerase repairs a target nucleic acid comprising one or more lesions during amplification of the target nucleic acid.

In certain embodiments, at least a portion of a target nucleic acid is amplified. In certain embodiments, the target nucleic acid to be amplified comprises one or more lesions. In certain embodiments, a composition comprising at least one lesion-repair polymerase, at least one second polymerase, at least one extendable nucleotide, at least one primer, and at least one target nucleic acid is formed. In certain embodiments, the composition is incubated under appropriate conditions to generate at least one primer extension product. In certain embodiments, the incubation is PCR. In certain embodiments, two primers are employed to amplify at least a portion of the target nucleic acid. In certain embodiments, multiple portions of the target nucleic acid may be amplified simultaneously to generate multiple primer extension products by employing multiple pairs of primers.

In certain embodiments, a lesion-containing target nucleic acid is incubated with at least one lesion-repair polymerase prior to a subsequent procedure. Exemplary subsequent procedures include, but are not limited to, amplification, sequencing, and genotyping. In certain embodiments, a lesion-containing target nucleic acid is amplified in the presence of a lesion-repair polymerase to generate at least one primer extension product. In certain embodiments, the lesion-repair polymerase is added during one or more incubations while amplifying a target nucleic acid. In certain embodiments, following amplification, a primer extension product may be used for sequencing, genotyping, further amplification, or other application. In certain embodiments, the subsequent sequencing, genotyping, further amplification, or other application may be in the presence or absence of a lesion-repair polymerase.

In certain embodiments, the sequence-of a nucleic acid may be determined by generating primer extension products. For example, in certain embodiments, one may employ the method of Sanger (see, e.g., Sanger et al. Proc. Nat. Acad. Sci 74: 5463-5467 (1977)). According to certain embodiments, methods are provided for sequencing a target nucleic acid using at least two polymerases. In certain embodiments, the methods employ a composition comprising at least one target nucleic acid, at least one primer, at least one extendable nucleotide, at least one terminator, and at least two polymerases. In certain embodiments, the at least two polymerases comprise at least one lesion-repair polymerase and at least one second polymerase. In certain embodiments, a duplex (double strandced polynucleotide) is formed between a target nucleic acid and a primer in the composition. In certain embodiments, the primer hybridizes to a predetermined location on the target nucleic acid.

In certain embodiments, the composition is incubated under appropriate reaction conditions, such that one or more extendable nucleotides are incorporated sequentially onto the 3′ end of the primer. In certain embodiments, a terminator may be incorporated into the primer extension product, and once incorporated, prevents further incorporation of nucleotides to the 3′ end of the primer extension product by polymerase. In certain embodiments, the primer extension products generated by the primer extension reaction may then be separated based on size. In certain embodiments, the sequence of the nucleic acid template may be determined from the particular sizes of the products and the identity of the terminator on each product.

In certain embodiments, a composition comprises at least two polymerases, at least one extendable nucleotide, and at least one terminator. In certain embodiments, the at least two polymerases comprise at least one lesion-repair polymerase and at least one second polymerase. In certain embodiments, the at least one extendable nucleotide is selected from dATP, dCTP, dITP, dGTP, dUTP, and dTTP. In certain embodiments, the composition comprises extendable nucleotides dATP, dCTP, dITP, and dUTP. In certain embodiments, the composition comprises extendable nucleotides dATP, dCTP, dITP, and dTTP. In certain embodiments, the at least one terminator is selected from an A terminator, a C terminator, a G terminator, and a T terminator. In certain embodiments, the at least one terminator further comprises a label. In certain embodiments, the at least one terminator further comprises an energy-transfer fluorescent dye (ETFD) label. In certain embodiments, the composition comprises an A terminator, a C terminator, a G terminator, and a T terminator. In certain embodiments, each of the different terminators further comprises a detectably different label. In certain embodiments, each of the different terminators further comprises a detectably different ETFD label. In certain embodiments, the composition contains four different ETFD-labeled terminators, e.g., an ETFD-labeled A terminator, an ETFD-labeled C terminator, an ETFD-labeled G terminator, and an ETFD-labeled T terminator, where each ETFD is detectably different.

In certain embodiments, a target nucleic acid comprising one or more lesions is incubated with at least one lesion repair polymerase prior to sequencing, genotyping, or amplification. In certain embodiments, a target nucleic acid comprising one or more lesions is incubated with at least one lesion repair polymerase during sequencing, genotyping, or amplification.

In certain embodiments, a composition further comprises at least one buffering agent. In certain embodiments, the at least one buffering agent is selected from Tris and Tricine. In certain embodiments, a composition further comprises at least one type of divalent cation. In certain embodiments, the at least one type of divalent cation is selected from Mg²⁺ and Mn²⁺. In certain embodiments, a composition further comprises at least one additive in certain embodiments, the at least one additive is selected from glycerol, DMSO, pTT, TAP, and BSA.

In certain embodiments, a composition comprises, in a 50 μL reaction volume, 15 mM Tris having a pH of 9.0, 2.5 mM MgCl₂, 200 μM. dATP, 200 μM dCTP; 200 μM dGTP, 200 μM dTTP, 2.5 U AmpliTaq® FS, 0.05-1.25 U lesion repair polymerase, 500 nM PCR primer, and an appropriate amount of a target nucleic acid including at least one lesion. In certain embodiments, the composition further includes at least one of DTT, glycerol, DMSO, TAP, and BSA.

In certain embodiments, a composition comprises, in a 20 μl reaction volume, 80 mM Tris having a pH in the range of 8-9; 5 mM MgCl₂; 0-10% glycerol; 200 μM dATP; 200 μM dCTP; 300 μM dITP; 200 μM dUTP; 25 nM-1225 nM of each of an ETFD-labeled A terminator, an ETFD-labeled C terminator, an ETFD-labeled G terminator, and an ETFD-labeled T terminator; and 1.5-60 units of each of at least two polymerases, wherein one of the polymerases is a lesion repair polymerase. In certain embodiments, the composition further comprises TAP. In certain embodiments, one uses the buffer, extendable nucleotides, and terminators from the ABI PRISM BigDye™ Terminators v. 3.0 Cycle Sequencing Kit (Applied Biosystems, Cat. No. 4390236), and replaces the kit's polymerase with at least one lesion-repair polymerase and at least one second polymerase. In certain embodiments, at least one of the at least one second polymerase is thermostable. In certain embodiments, the at least one second polymerase comprises Taq (G46D; F667Y; E681I) and Taq (G46D; F667Y; T664N; R660G).

In certain embodiments, methods are provided for sequencing a target nucleic acid. In certain embodiments, the target nucleic acid comprises one or more lesions. In certain embodiments, such methods comprise forming a composition comprising a target nucleic acid, at least one primer, at least one extendable nucleotide, at least one terminator, at least one lesion-repair polymerase, and at least one second polymerase. In certain embodiments, at least one of the at least one second polymerase is thermostable. In certain embodiments, the at least one second polymerase comprises Taq (G46D; F667Y; E681I) and Taq (G46D; F667Y; T664N; R660G). In certain embodiments, the method comprises incubating the composition under appropriate conditions to generate at least one primer extension product.

In certain embodiments, the methods include cycle sequencing, in which, following the primer extension reaction and termination, the primer extension product is released from the target nucleic acid, and a new primer is annealed, extended, and terminated. Cycle sequencing is but one example of amplification of primer extension products. In certain embodiments, cycle sequencing is performed using a thermocycler apparatus. Certain cycle sequencing reactions are described, e.g., in U.S. Pat. Nos. 5,741,640; 5,741,676; 5,756,285; 5,674,679; and 5,998,143.

In cycle sequencing, an incubation cycle comprises two or more incubations, each incubation comprising a certain temperature for a certain period of time. In certain embodiments, one such incubation cycle comprises 95° C. for 20 seconds, followed by 50° C. for 15 seconds, followed by 60° C. for 4 minutes. In certain embodiments, cycle sequencing comprises repeating the incubation cycle 25 times.

In certain embodiments, the primer extension products may be separated by a mobility-dependent analysis technique, or MDAT. In certain embodiments, the MDAT is electrophoresis. In certain embodiments, by separating the primer extension products, one can determine the sequence of the template nucleic acid based on the size of each product and the identity of the terminator at its 3′ end. In certain embodiments, when the terminator is a labeled terminator, the identity of the terminator at the 3′ end is determined by the identity of the label.

In certain embodiments, one may use the lesion repair polymerase compositions of the invention in forensic applications. In the area of forensics, in certain instances, identification of DNA-containing samples can be hindered by degradation of the samples {see, e.g., Butler et al., J Forensic Sci., 48(5): 1054-1064 (2003); Grubwieser et al., Int J Legal Med., 117(3): 185-188 (2003); Wiegand et al., Int J Legal Med., 114(4-5): 285-287 (2001); Tsukada et al., Leg Med. (Tokyo)., 4(4): 239-245 (2002); Hellmann et al., Int J Legal Med., 114(4-5): 269-273 (2001)). In certain instances, identification of DNA-containing samples can be hindered by the presence of lesions in the samples. In various embodiments, identification of DNA-containing samples is important in forensic applications for the identification of human remains, for disaster and military victim identification (see, e.g., Fre'geau et al. (1993) Biotechniques 15:100-119), for the analysis of museum specimens, for the identification of criminals, and for parentage testing. In certain embodiments, compositions and methods may be used to amplify degraded DNA samples, thereby assisting in the identification of DNA-containing samples. In certain embodiments, compositions and methods may be used to amplify any one or more of the following marker loci in degraded DNA samples: THO1, AMG, D8, FGA, D3, D16, D18, TPOX, CSF, D19, D21, D7, D5, D13, D2, vWA, and loci described in the Short Tandem Repeat DNA Internet Database (available at http://www.cstl.nist.gov/biotech/strbase/, last accessed Dec. 15, 2003).

In various embodiments, the methods can be performed on DNA extracted from various specimens that contain nucleic acid, e.g., bone, hair, blood, and tissue. In certain embodiments, DNA may be extracted from a specimen and a panel of primers may be used to amplify a set of microsatellites to generate a set of amplified fragments. In certain embodiments, the set of amplified fragments is separated on the basis of mobility to generate a microsatellite amplification pattern. In certain embodiments, the specimen's microsatellite amplification pattern is compared to the microsatellite amplification pattern of a known sample. In certain embodiments, the known sample is a sample presumed to be the same as the specimen's (sometimes referred to as the “presumptive specimen”). In certain embodiments, the known sample is a sample from a family member of the presumptive specimen. In certain embodiments, the same set of microsatellites is amplified in each sample.

In certain embodiments, the pattern of microsatellite amplification pattern is used to confirm or rule out the identity of the specimen. In certain embodiments applicable to paternity testing, microsatellite amplification patterns are used to confirm or rule out the identity of the father. In certain embodiments, the microsatellite amplification pattern of a child is compared to the microsatellite amplification pattern of the presumptive father. In certain embodiments, the microsatellite amplification pattern of the child is also compared to the microsatellite amplification pattern of the child's mother.

In certain embodiments, a microsatellite amplification pattern is derived from amplification of one or more microsatellites. In certain embodiments, microsatellites with a G+C content of 50% or less are used. Exemplary microsatellites with a G+C content of 50% or less include, but are not limited to, D3S1358; vWA; D16S539; D8S1179; D21S11; D18S51; D19S433; TH01; FGA; D7S820; D13S317; D5S818; CSF1PO; TPOX; hypoxanthine phosphoribosyltransferase; intestinal fatty acid-binding protein; recognition/surface antigen; c-fms proto-oncogene for CFS-1 receptor; tyrosine hydroxylase; pancreatic phospholipase A-2; coagulation factor XIII; aromatase cytochrome P-450; lipoprotein lipase; c-fes/fps proto-oncogene. In various embodiments, one or more microsatellites selected from D3S1 358; vWA; D16S539; D8S1179; D21S11; D18S51; D19S433; THO1; FGA; D7S820; D13S317; D5S818; CSF1 PO; and TPOX are used for paternity, forensic, and/or other personal identification.

According to certain embodiments, kits are provided. In certain embodiments, kits serve to expedite the performance of the methods of interest by assembling two or more components used to carry out the methods. In certain embodiments, kits contain components in pre-measured unit amounts to minimize the need for measurements by end-users. In certain embodiments, kits include instructions for performing one or more methods. In certain embodiments, the kit components are optimized to operate in conjunction with one another.

In certain embodiments, kits comprise at least one lesion repair polymerase and at least one second polymerase. In certain embodiments, the at least one second polymerase comprises at least one of Taq (G46D; F667Y; E681I) and Taq (G46D; F667Y; T664N; R660G). In certain embodiments, kits comprise three or more polymerases, at least one of which is a lesion repair polymerase.

In certain embodiments, a kit may be used to amplify at least one target nucleic acid. In certain embodiments, a kit may be used to amplify at least one lesion-containing target nucleic acid. In certain embodiments, a kit may be used to genotype a target nucleic acid. In certain embodiments, a kit may comprise additional components, including, but not limited to, at least one primer, at least one probe, and/or at least one extendable nucleotide.

In certain embodiments, a kit may be used to sequence at least one target nucleic acid. In certain embodiments, a kit further comprises at least one terminator. In certain embodiments, the at least one terminator is a labeled terminator. In certain embodiments, the at least one terminator is selected from an ETFD-labeled A terminator, an ETFD-labeled C terminator, an ETFD-labeled G terminator, and an ETFD-labeled T terminator.

In certain embodiments, a kit may also comprise reagents for performing a control reaction, which may include one or more of the above components, and at least one target nucleic acid.

Other embodiments of the invention will be apparent to those skilled in the art from consideration of the specification. It is intended that the specification and examples be considered as exemplary only, with the true scope and spirit of the invention being indicated by the following claims.

1. A composition comprising at least one lesion repair polymerase and at least one second polymerase.

2. The composition of claim 1, wherein the at least one second polymerase is not a lesion repair polymerase.

3. The composition of claim 1, wherein at least one of the at least one second polymerase is thermostable.

4. The composition of claim 3, wherein at least one of the at least one lesion repair polymerase is thermostable.

5. The composition of claim 1, wherein at least one of the at least one lesion repair polymerase is an X family polymerase.

6. The composition of claim 5, wherein the X family polymerase is selected from DNA polymerase β, DNA polymerase λ, DNA polymerase σ, DNA polymerase μ, DpoB, TDT, and ASFV polymerase X.

7. The composition of claim 1, wherein at least one of the at least one lesion repair polymerase is a Y family polymerase.

8. The composition of claim 7, wherein the Y family polymerase is selected from DNA polymerase η, DNA polymerase I, DNA polymerase κ, Rev 1, Rad 30, DinB, UmuC, UmuD2C, UmuD′2C, Dpo4, Dbh, and bacterial DNA pol II.

9. The composition of claim 1, wherein the at least one lesion repair polymerase is one lesion repair polymerase and wherein the at least one second polymerase is one second polymerase.

10. The composition of claim 9, wherein the lesion-repair polymerase and the second polymerase are present at a weight ratio from 1:4999 to 1:99.

11. The composition of claim 9, wherein the lesion-repair polymerase and the second polymerase are present at a weight ratio of 1:99 to 50:50.

12. The composition of claim 9, wherein the lesion-repair polymerase and the second polymerase are present at a weight ratio from 50:50 to 99:1.

13. The composition of claim 9, wherein the lesion-repair polymerase and the second polymerase are present at a unit ratio from 1:4999 to 1:99.

14. The composition of claim 9, wherein the lesion-repair polymerase and the second polymerase are present at a unit ratio from 1:99 to 50:50.

15. The composition of claim 9, wherein the lesion-repair polymerase and the second polymerase are present at a unit ratio from 50:50 to 99:1.

16. The composition of claim 1, wherein the at least one lesion-repair polymerase is one lesion-repair polymerase and wherein the at least one second polymerase is two second polymerases.

17. The composition of claim 16, wherein at least one of the two second polymerases is thermostable.

18. The composition of claim 17, wherein the two second polymerases are Taq (G46D; F667Y; E681I) and Taq (G46D; F667Y; T664N; R660G).

19. The composition of claim 18, wherein the unit ratio of Taq (G46D; F667Y; E681I) and Taq (G46D; F667Y; T664N; R660G) is from 1:99 to 50:50.

20. The composition of claim 18, wherein the unit ratio of Taq (G46D; F667Y; E681I) and Taq (G46D; F667Y; T664N; R660G) is from 50:50 to 99:1.

21. The composition of claim 16, wherein the weight ratio of the lesion-repair polymerase to the two second polymerases is from 1:4999 to 1:99.

22. The composition of claim 16, wherein the weight ratio of the lesion-repair polymerase to the two second polymerases is from 1:99 to 50:50.

23. The composition of claim 16, wherein the weight ratio of the lesion-repair polymerase to the two second polymerases is from 50:50 to 99:1.

24. The composition of claim 16, wherein the unit ratio of the lesion-repair polymerase to the two second polymerases is from 1:4999 to 1:99.

25. The composition of claim 16, wherein the unit ratio of the lesion-repair polymerase to the two second polymerases is from 1:99 to 50:50.

26. The composition of claim 16, wherein the unit ratio of the lesion-repair polymerase to the two second polymerases is from 50:50 to 99:1.

27. The composition of claim 1, further comprising a target nucleic acid.

28. The composition of claim 27, wherein the target nucleic acid is a lesion-containing target nucleic acid.

29. The composition of claim 1, further comprising at least one primer and at least one extendable nucleotide.

30. The composition of claim 28, further comprising at least one of a terminator, a buffering agent, and an additive.

31. A method of amplifying a lesion-containing target nucleic acid comprising incubating the lesion-containing target nucleic acid with at least one primer, at least one extendable nucleotide, at least one lesion-repair polymerase, and at least one second polymerase under conditions to generate at least one primer extension product.

32.-56. (canceled)

57. A method of sequencing a lesion-containing target nucleic acid comprising:
(a) incubating the lesion-containing target nucleic acid with at least one primer, at least one extendable nucleotide, at least one terminator, at least one lesion repair polymerase, and at least one second polymerase, under conditions to generate at least one primer extension product comprising a terminator;
(b) separating the at least one primer extension product comprising a terminator;
(c) detecting at least one of the at least one primer extension product comprising a terminator; and
(d) determining the sequence of the lesion-containing target nucleic acid.

58.-84. (canceled)

85. A method of genotyping a lesion-containing target nucleic acid comprising:
(a) incubating the lesion-containing target nucleic acid with at least one primer, at least one extendable nucleotide, at least one lesion repair polymerase, and at least one second polymerase, under conditions to generate at least one primer extension product;
(b) separating the at least one primer extension product;
(c) detecting the at least one primer extension product; and
(d) determining the genotype of the lesion-containing target nucleic acid.

86.-194. (canceled)

195. A kit comprising at least one lesion repair polymerase and at least one second polymerase.

196. The kit of claim 195, wherein at least one of the at least one lesion repair polymerase is an X family polymerase.

197. The kit of claim 196, wherein the X family polymerase is selected from DNA polymerase β, DNA polymerase λ, DNA polymerase σ, DNA polymerase μ, DpoB, TDT, and ASFV polymerase X.

198. The kit of claim 195, wherein at least one of the at least one lesion repair polymerase is a Y family polymerase.

199. The kit of claim 198, wherein the Y family polymerase is selected from DNA polymerase η, DNA polymerase I, DNA polymerase κ, Rev 1, Rad 30, DinB, UmuC, UmuD2C, UmuD′2C, Dpo4, Dbh, and bacterial DNA pol II.

200. The kit of claim 195, wherein at least one of the at least one second polymerase is thermostable.

201. The kit of claim 195, wherein the at least one second polymerase is two second polymerases.

202. The kit of claim 201, wherein at least one of the two second polymerases is thermostable.

203. The kit of claim 202, wherein the two second polymerases are Taq (G46D; F667Y; E681I) and Taq (G46D; F667Y; T664N; R660G).

204. The kit of claim 195, further comprising at least one of a terminator, a buffering agent, a divalent cation, and an additive.
